SEQUENCE LISTING (24 sequences)

SEQ ID NO: 1 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 2 - protein, 61 aa, Homo sapiens
GRTCPKPDDLPFSTVVPLKTFYEPGEEITYSCKPGYVSRGGMRKFICPLTGLWPINTLKCT

SEQ ID NO: 3 - protein, 58 aa, Homo sapiens
PRVCPFAGILENGAVRYTTFEYPNTISFSCNTGFYLNGADSAKCTEEGKWSPELPVCA

SEQ ID NO: 4 - protein, 63 aa, Homo sapiens
PIICPPPSIPTFATLRVYKPSAGNNSLYRDTAVFECLPQHAMFGNDTITCTTHGNWTKLPECR

SEQ ID NO: 5 - protein, 60 aa, Homo sapiens
EVKCPFPSRPDNGFVNYPAKPTLYYKDKATFGCHDGYSLDGPEEIECTKLGNWSAMPSCK

SEQ ID NO: 6 - protein, 84 aa, Homo sapiens
ASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 7 - protein, 242 aa, Homo sapiens
GRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCK

SEQ ID NO: 8 - protein, 326 aa, Homo sapiens
GRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 9 - DNA, 27 bp, artificial sequence
  primer
gtactagtggacggacctgtcccaagc

SEQ ID NO: 10 - DNA, 30 bp, artificial sequence
  synthetic
gaggtacctgtacatttcagagtgttgatg

SEQ ID NO: 11 - DNA, 29 bp, artificial
  synthetic sequence
gtactagtgcatcttgtaaagtacctgtg

SEQ ID NO: 12 - DNA, 28 bp, artificial sequence
  synthetic
gaggtaccgcatggctttacatcggatg

SEQ ID NO: 13 - DNA, 29 bp, artificial sequence
  synthetic
gccactagtgaagtaaaatgcccattccc

SEQ ID NO: 14 - DNA, 35 bp, artificial sequence
  synthetic
gaggtacctttacaacttggcatggcagaccagtt

SEQ ID NO: 15 - DNA, 29 bp, artificial sequence
  synthetic
gtgctagcatgatttctccagtgctcatc

SEQ ID NO: 16 - DNA, 57 bp, artificial sequence
  synthetic
gaattcctacttgtcatcgtcatccttgtagtctgtacatttcagagtgttgatggg

SEQ ID NO: 17 - DNA, 29 bp, artificial sequence
  synthetic
gtgctagcatgatttctccagtgctcatc

SEQ ID NO: 18 - DNA, 51 bp, artificial sequence
  synthetic
gaattcctacttgtcatcgtcatccttgtagtcagcacagacaggaagctc

SEQ ID NO: 19 - DNA, 29 bp, artificial sequence
  synthetic
gtgctagcatgatttctccagtgctcatc

SEQ ID NO: 20 - DNA, 56 bp, artificial sequence
  synthetic
gaattcctacttgtcatcgtcatccttgtagtccctgcattctggtaatttagtcc

SEQ ID NO: 21 - DNA, 29 bp, artificial sequence
  synthetic
gtgctagcatgatttctccagtgctcatc

SEQ ID NO: 22 - DNA, 56 bp, artificial sequence
  synthetic
gaattcctacttgtcatcgtcatccttgtagtctttacaacttggcatggcagacc

SEQ ID NO: 23 - DNA, 29 bp, artificial sequence
  synthetic
gtgctagcatgatttctccagtgctcatc

SEQ ID NO: 24 - DNA, 54 bp, artificial sequence
  synthetic
gaattcctacttgtcatcgtcatccttgtagtcgcatggctttacatcggatgc

(19) United States
(12) Patent Application Publication
(10) Pub. No.: US 2018/0085454 A1
(43) Pub. Date: Mar. 29, 2018

(54) RECOMBINANT beta2-GPI PEPTIDES AND THE USE THEREOF IN ANTI-TUMOR THERAPY

(71) Applicant: NATIONAL YANG-MING UNIVERSITY, Taipei (TW)

(72) Inventors: Anna CHIANG, Taipei (TW); Shr-Jeng LEU, Taipei (TW)

(21) Appl. No.: 15/270,630

(22) Filed: Sep. 20, 2016

(30) Foreign Application Priority Data
Jan. 20, 2016 (TW) 105101747

(51) Int. Cl. A61K 39/395; C07K 16/18; C12N 15/63

(52) U.S. Cl. CPC A61K 39/3955; A61K 2039/505; C12N 15/63; C07K 16/18

(57) ABSTRACT

A purified recombinant β₂-GPI peptide having at least one tumor inhibitory β₂-GPI domain, and the use thereof in anti-tumor therapy. Producing and purifying various recombinant β₂-GPI fragments using a viral expression system; screening functional domains of β₂-GPI with potential in inhibiting tumor cell proliferation and migration; preparing purified recombinant β₂-GPI peptides having the functional domains including β₂-GPI-D1, β₂-GPI-D4, β₂-GPI-D5, β₂-GPI-D1234, β₂-GPI-D12345 and β₂-GPI-D2345; and applying the purified recombinant β₂-GPI peptides in the development of anti-tumor protein drugs or related therapy.

### BACKGROUND OF THE INVENTION

### Technical Field of the Invention

The present invention relates to purified recombinant β₂-GPI peptides, and their use in anti-tumor therapy. Especially, the present invention relates to a recombinant β₂-GPI peptide produced by using a virus expression system, comprising at least one functional fragment of β₂-GPI exhibiting inhibitory activities on tumor cell proliferation, migration and invasion.

### Background

β₂-glycoprotein I (β₂-GPI) is a plasma glycoprotein with diverse physiological functions in human body. β₂-GPI has a molecular weight of −50 kDa and is composed of 326 amino acids with the sequence as listed in SEQ ID No. 1. The amino acid sequence of β₂-GPI protein contains 5 domains, each of the domains 1 to 4 has about 60 amino acids and the domain 5 has about 80 amino acids. The first four domains each contains two pair of disulfide bonds, forming a structure called short consensus repeat (SCR) or complement control protein repeat (CCP) (Kristensen, T. et al., FEBS Lett 289(2): p. 183-6, 1991).

The published literatures indicate that β₂-GPI is found in atherosclerotic plaques, which suggests that β₂-GPI may be related to the formation of atherosclerosis (Ross, R., Am Heart J 138(5 Pt 2): p. S419-20, 1999; Weber, C., A. Zernecke and P. Libby, Nat Rev Immunol 8(10): p. 802-15, 2008). Previous studies have shown that β₂-GPI exhibits protective effects of inhibiting low density lipoprotein (LDL) oxidation and reducing the endocytosis of cholesterol by macrophage (Lin, K. Y. et al., Life Sci 69(6): p. 707-19, 2001). However, other literatures have found that β₂-GPI will bind to oxidized low density lipoprotein (ox-LDL), and exacerbated atherosclerosis process when coupled with the patient's anti-APS-β₂-GPI antibodies (Liu, Q. et al., J Lipid Res 43(9): p. 1486-95, 2002). Therefore, the role of β₂GPI in the progress of atherosclerosis is still controversial.

Recently, it has been reported that the of clipped form of β₂-GPI after the plasmin digestion at Lys317-Lys318 exhibits suppressive activities on angiogenesis (Beecken, W. D. et al., Cancer Lett 296(2): p. 160-7, 2010.; Sakai, T. et al., Am J Pathol 171(5): p. 1659-69, 2007), and that the phenomenon angiogenesis is aggravated in β₂-GPIgene knock-out mice (Passam, F. H. et al., J Autoimnmun 35(3): p. 232-40, 2010). US patent application no. 2013/0165391 disclosed a peptide fragment 296Cys-Ser345 (from amino acid no. 269 to 345) derived from the domain V of β₂-GPI with the ability to reduce tumor volume in melanoma and breast cancer, and alleviate the angiogenesis caused by melanoma and breast cancer.

When tumor cells grow to a size exceeding 3 mm³ in their primary site, nutrients provided by the surrounding tissue will be insufficient for the provide growth of tumor cells. The tumor cells secrete growth factors stimulating angiogenesis in surrounding tissue to the tumor site to provide sufficient nutrients for continues growth. Therefore, there are close relationships between angiogenesis and tumor growth. Literatures have indicated that plasma proteins having activities to modulate angiogenesis, such as angiostatin, endostatin and thrombospondin, can not only affect physiological functions of angiogenesis, but also play a role in tumor growth and metastasis in experimental mice (Cui, R. et al., Cancer Sci 98(6): p. 830-7, 2007; Gonzalez-Gronow, M. et al., Exp Cell Res 303(1): p. 22-31, 2005; O'Reilly, M. S. et al., Cell 88(2): p. 277-85, 1997; Reiher, F. K. et al., Int J Cancer 98(5): p. 682-9, 2002).

Our prior studies have shown that β₂-GPI can suppress melanoma cell growth and migration by inhibiting Akt phosphorylation and activation of NF-κB pathway. It has been found that purified β₂-GPI could inhibit tumor cell growth in mice subcutaneously implanted melanoma cells. These results suggest that β2-GPI plays a negative role in angiogenesis, and exhibits effects on the tumor progression.

In 2012, our lab found that purified β₂-GPI could suppress vascular endothelial growth factor (VEGF)-induced cell growth and migration in human aortic endothelial cells (HAECs). We also found that purified β₂-GPI could inhibit the cell migration in the A375 and B16-F10 melanoma cells. The proliferation and migration play important roles in the tumor progression (Fearon, E R et al., Cell 61(5): p 759-67, 1990; Wood L D et al., Science 318(5853): p 1108-13. 2007; Guan X et al., Acta Pharm Sin B. 5(5): p 402-18, 2015). So, we propose to clarify the functional domain of β₂-GPI in anti-tumor cell migration and proliferation, and explore the therapeutic potential of recombinant β₂-GPI peptides for anti-tumor application.

### SUMMARY OF INVENTION

In the present invention, various β₂-GPI peptide fragments are designed and expressed in viral and eukaryotic expression systems in order to identify the functional domains of β₂-GPI having the biological activities of anti-tumor cell migration and anti-tumor cell proliferation in vitro as well as anti-tumor growth in vivo.

Accordingly, in one aspect, the present invention relates to a purified recombinant β₂-GPI peptide, comprising at least one functional β₂-GPI peptide fragment exhibiting anti-tumor activities.

In certain embodiments of the present invention, the functional β₂-GPI peptide fragment is selected from a group consisted of the domain 1 (D1, SEQ ID No. 2), domain 2 (D2, SEQ ID No. 3), domain 3 (D3, SEQ ID No. 4), domain 4 (D4, SEQ ID No. 5), domain 5 (D5, SEQ ID No. 6) of β₂-GPI protein. In some embodiments, the purified recombinant β₂-GPI peptide comprises a functional β₂-GPI-D1 peptide fragment. In other embodiments, the purified recombinant β₂-GPI peptide comprises a functional β₂-GPI-D4 peptide fragment.

In another aspect, the present invention relates to a pharmaceutical composition for suppressing tumors, comprising the said purified recombinant β₂-GPI peptide. In one embodiment of the present invention, the recombinant β₂-GPI peptide comprises a β₂-GPI domain 1 (D1) fragment, so called as β₂-GPI-D1 peptide fragment. In another embodiment of the present invention, the recombinant β₂-GPI peptide comprises a β₂-GPI domain 4 (D4) fragment, so called as β₂-GPI-D4 peptide fragment.

In a further embodiment of the present invention, the recombinant β₂-GPI peptide comprises a β₂-GPI domain 1-4 (D1-4) fragment, so called as β₂-GPI-D1234 peptide fragment (SEQ ID No. 7). In yet another embodiment of the present invention, the recombinant β₂-GPI peptide comprises a β₂-GPI domain 1-5 (D1-5) fragment, so called as β₂-GPI-D12345 peptide fragment (SEQ ID No. 8).

In certain embodiments of the present invention, the pharmaceutical composition is used to inhibit tumor cell growth. In other embodiments of the present invention, the pharmaceutical composition is used to inhibit angiogenesis in a tumor progressing site. In other embodiments of the present invention, the pharmaceutical composition is used to inhibit tumor metastasis.

### BRIEF DESCRIPTION OF THE DRAWINGS

FIG. 1 shows the design of various recombinant β₂-GPI constructs for cloning into the plasmid pFastBac1. The constructs contain the DNA sequences of a Honeybee Melittin Secretion (MS) Signal peptide, dual Flag/StrepII affinity tag, Tabacco Etch Virus (TEV) cleavage site, and human IgG1 Fc fusion protein (Fc).

FIG. 2 shows the constructs in the multiple cloning site (MCS) of the recombinant viral vectors for expressing recombinant human β₂-GPI-D1-Fc, β₂-GPI-D4-Fc, β₂-GPI-D5-Fc, β₂-GPI-D1234-Fc and β₂-GPI-D12345-Fc peptides.

FIG. 3 shows the Western blot analysis of the recombinant β₂-GPI-D1-Fc, β₂-GPI-D4-Fc, β₂-GPI-D5-Fc, β₂-GPI-D1234-Fc and β₂-GPI-D12345-Fc peptides expressed in Sf9 cells infected with recombinant Baculovirus. Conditional medium of Sf9 cells were collected and assayed directly by SDS-PAGE and then analyzed by Western blot using anti-strep monoclonal antibody.

FIG. 4 shows the purification of the recombinant human β₂-GPI-D1-Fc, β₂-GPI-D4-Fc, β₂-GPI-D5-Fc, β₂-GPI-D1234-Fc and β₂-GPI-D12345-Fc peptides. The purity and yield of the recombinant proteins were determined by 12% SDS-PAGE.

FIGS. 5A and 5B show the purification and identification of human β₂-GPI isolated from healthy human plasma by Heparin-Sepharose affinity chromatography. FIG. 5A shows the UV absorbance spectra of β₂-GPI purification fractions at 280 nm. The purified β₂-GPI was collected in fractions of peak III. FIG. 5B shows the purity of β₂-GPI determined by SDS-PAGE and western blot analysis, a single band above molecular weight of 55 k Da is showed on the western blot.

FIGS. 6A-6C show the effects of different peptide domains of β₂-GPI on B16-F10 cell viability and proliferation in B16-F10 melanoma cells assessed by MTT assay (FIG. 6A), BrdU proliferation assay (FIG. 6B) and counting the cell number (FIG. 6C). Results are means±SEM of at least three independent experiments. * P<0.05, **P<0.01, ***P<0.001 versus control.

FIG. 7 shows the effects of different peptide domains of β₂-GPI on B16-F10 cell migration were evaluated by using a modified Boyden chamber assay. Results are means±SEM of at least three independent experiments. ** P<0.01 versus control. ***P<0.001 versus control.

FIG. 8 shows the effects of plasma purified β₂-GPI at 25, 50, 100, 200 g/ml on B16-F10 cell invasion by using a Matrigel invasion assay. Bovine serum albumin was used as a control protein. Results are means±SEM. of at least three independent experiments. ***P<0.001 versus control.

FIG. 9A shows the effects of recombinant β₂-GPI-D1, β₂-GPI-D4, β₂-GPI-D5, Pβ₂-GPI-D1234 and β₂-GPI-D12345 peptides on cell migration in B16-F10 melanoma cells evaluated by a wound-healing assay. FIG. 9B is the bar graph of wound areas (wound areas of 0 hr−wound areas of 24 hr) calculated and presented as percentage of control. Results are means±S.E.M. of at least three independent experiments. ***P<0.001 compared with the control by ANOVA followed by the Dunnett's Multiple Comparison Test.

FIG. 10 shows the effects of peptide domains of β₂-GPI on B16-F10 cell migration evaluated by a Matrigel invasion assay. Results are means±SEM. of at least three independent experiments. ***P<0.001 versus control.

FIG. 11 shows the design of five recombinant β₂-GPI constructs, β₂-GPI-D1-Flag, β₂-GPI-D12-Flag, β₂-GPI-D123-Flag, β₂-GPI-D1234-Flag and β₂-GPI-D12345-Flag, for being expressed in Lentivirus expression system.

FIGS. 12A and 12B show the cellular proteins of A375 human melanoma cells (FIG. 12A) and B16-F10 mouse melanoma cells (FIG. 12B) infected by lentivirus packaged with pLKO AS3w.puro vector control and pLKO AS3w.puro β₂-GPI constructs analyzed by western blot. Host cells without virus infection were shown as the control (C). V means melanoma cells infected by virus with vector alone.

FIGS. 13A and 13B show the effects of domain I of β₂-GPI inhibiting tumor growth in B16-F10-implanted C57BL/6 mice. FIG. 13A shows the change of tumor volume during the treatment of PBS (control), β₂-GPI, β₂-GPI-D12345, β₂-GPI-D1 or Fc. Results are presented as means±SEM. *** P<0.001 compared with PBS by ANOVA followed by Dunnett's Multiple Comparison Test. FIG. 13B shows the tumor weight measured at the end of the experiment. Results are presented as medium and interquartile range (IQR). P values are shown in the graph; difference between groups was compared using ANOVA followed by Dunnett's Multiple Comparison Test.

### DETAILED DESCRIPTION OF THE INVENTION

As used herein, “purified recombinant β₂-GPI peptide” relates to a recombinant β₂-GPI protein or peptide comprising at least one functional fragment of β₂-GPI with anti-tumor activities, expressed and purified from an expression system.

As used herein, “functional fragment of β₂-GPI with anti-tumor activities” relates to a β₂-GPI peptide fragment exhibiting inhibitory activities on inhibiting the growth, proliferation, migration of tumor cells and the angiogenesis in tumor progressing site, which have been determined by in vitro or in vivo tests. The functional fragment of β₂-GPI of present invention includes, but is not limited to, the domain 1 (D1, SEQ ID No. 2), domain 2 (D2, SEQ ID No. 3), domain 3 (D3, SEQ ID No. 4), domain 4 (D4, SEQ ID No. 5), domain 5 (D5, SEQ ID No. 6), and their combination with each other, of β₂-GPI protein.

As used herein, “polypeptide”, “peptide” and “protein” are used interchangeably and include reference to a polymer of amino acid residues.

As used herein, the term “recombinant” refers to a protein produced using cells that do not have, in their native state, an endogenous copy of the DNA able to express the protein. The cells produce the recombinant protein because they have been genetically altered by the introduction of the appropriate isolated nucleic acid sequence.

“Expression system” or “expression vector” refers to nucleic acid sequences containing a desired coding sequence and control sequences in operable linkage, so that hosts transformed with these sequences are capable of producing the encoded proteins. To effect transformation, the expression system may be included on a vector; however, the relevant nucleic acid molecule may then also be integrated into the host chromosome.

Exemplary vectors used in the methods of the invention include a plasmid, a cosmid or a viral vector. A suitable expression vector can be prepared from genomic or cDNA encoding β₂-GPI peptide fragment, that is optionally under the control of a suitable operably connected inducible promoter, enhancer or other expression controlling elements, such as T7 promoter, CaMV 35S promoter, Simian Virus 40 early or late promoter, Rous sarcoma virus long terminal repeat promoter, cytomegalovirus promoter, adenovirus late promoter, glycolipid anchored surface protein (GAS) signal peptide or polyadenylation signals.

The host cells employed in the methods of the invention can include E. coli, B. subtilis, a Saccharomyces eukaryotic host cell, an insect eukaryotic host cell (e.g., at least one member selected from the group consisting of a Baculovirus infected insect cell, such as Spodoptera frugiperda (Sf9) or Trichoplusia ni (High5) cells), a fungal eukaryotic host cell, a parasite eukaryotic host cell (e.g., a Leishmania tarentolae eukaryotic host cell), CHO cells, yeast cells (e.g., Pichia) and a Kluyveromyces lactis host cell.

The recombinant β₂-GPI peptide or fusion protein produced by the method of present invention is useful for preparing an anti-tumor composition having effects on preventing tumorigenesis, metastasis, inhibiting tumor cell proliferation, migration and/or angiogenesis in a subject which is susceptible to or suffering from cancers. The composition may be produced by a method described herein or other techniques known in the pharmaceutical art.

For example, the recombinant β₂-GPI peptides can be formulated with a pharmaceutically acceptable carrier, such as a phosphate buffer or a bicarbonate buffer for oral or parenteral administration. The carrier must be “acceptable” in the sense that it is compatible with the active ingredient of the composition, and preferably, capable of stabilizing the active ingredient and not deleterious to the subject to be treated. The carrier is selected on the basis of the mode and route of administration, and standard pharmaceutical practice. Suitable pharmaceutical carriers and diluents, as well as pharmaceutical necessities for their use, are described in Remington's Pharmaceutical Sciences.

### EXAMPLES

The other characteristics and advantages of the present invention will be further illustrated and described in the following examples. The examples described herein are using for illustrations, not for limitations of the invention.

### Example 1. Preparation of Recombinant β₂-GPI Peptides in Baculovirus Expression System

Preparation of the Bac-to-Bac Baculovirus Expression System

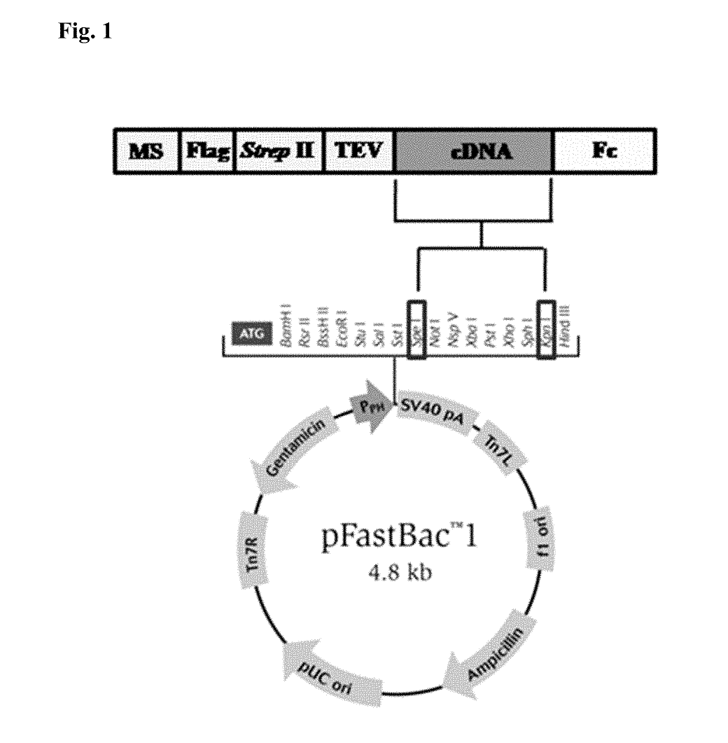

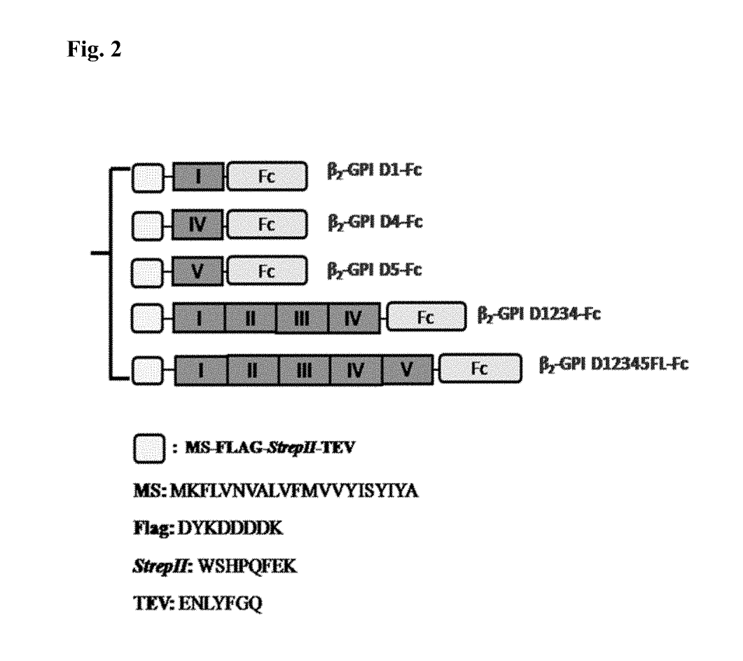

At first, the recombinant β2-GPI gene fragments comprising β2-GPI-D12345, β2-GPI-D1234, β2-GPI-D2345, β2-GPI-D1, β₂-GPI-D4 or βP2-GPI-D5 coding sequence were firstly constructed into pFastBac1 plasmids as listed in FIG. 1. The constructs for expressing human β₂-GPI-D1-Fc, β₂-GPI-D4-Fc, β₂-GPI-D5-Fc, β₂-GPI-D1234-Fc and β₂-GPI-D12345-Fc recombinant peptides were cloned in the multiple cloning site (MCS) of the Baculovirus expression vector are showed in FIG. 2.

The plasmid pFastBac1-NT-FLAG was digested with restriction enzymes SpeI and KpnI, the plasmid DNA was isolated by gel extraction. The various DNA inserts comprising domains D1 to D5 of β₂-GPI were amplified by PCR using the primers β2-GPI-SpeI-s(Bac) (ments, SEQ ID No. 9), β₂-GPI-D1-KpnI-a(Bac) (GAGGTACCTGTACATTTCAGAGTGTTGATG, SEQ ID No. 10), β₂-GPI-D5-SpeI-s(Bac) (GTACTAGTGCATCTTGTAAAGTACCTGTG, SEQ ID No. 11), β₂-GPI-KpnI-a(Bac) (GAGGTACCGCATGGCTTTACATCGGATG, SEQ ID No. 12), β₂-GPI-D4-SpeI-a(Bac) (GCCACTAGTGAAGTAAAATGCCCATTCCC, SEQ ID No. 13) or β₂-GPI-D4-KpnI-a(Bac) (GAGGTACCTTTACAACTTGGCATGGCAGACCAGTT, SEQ ID No. 14). The PCR products were ligated to a TA plasmid, and cut from the TA plasmid with the digestion of restriction enzymes SpeI and KpnI. The inserts were ligated to the digested NPFastBac1-NT-FLAG vector to form recombinant Baculovirus. The ligated vectors were transformed into the DH10Bac competent cells. The Bacmid DNA was isolated in small scale for confirming the successful ligation of the inserts into the vector NPFastBac1-NT-FLAG by restriction enzyme digestions, and for further identifying DNA sequencing.

The prepared recombinant pFastBac1 plasmids carrying the coding sequence of various β2-GPI peptide fragments were transfected into the Sf9 insect cells pre-cultured in a 6-well cell culture dish at the day before the experiment. Briefly, the sf9 insect cells were inoculated in a 6-well cell culture dish (to about 50% confluent), and cultured at 27° C. for 16 hours. 100 μl of serum-free medium containing 1 μg of the isolated Bacmid DNA was added to a solution of 6 μl of Cellfectin and 100 μl of serum-free medium in an eppendorf tube, and mixed thoroughly. The mixed solution was stand at room temperature (RT) for 30 min, and 0.8 ml of serum-free medium was added to the solution and mixed thoroughly. Then, the mixed solution was added to the pre-cultured Sf9 insect cells, incubated at 27° C. for 6-8 hours to carry out transfection. The transfected cell were transferred to 2 ml of medium with supplementary serum, and cultured at 27° C. for 7 days. The supernatant containing recombinant baculovirus was collected by centrifuging the cell culture at 4° C. at 500×g for 10 min, and stored at 4° C.

For the large scaled production of recombinant β₂-GPI peptide expressing virus, the obtained supernatant containing recombinant baculovirus was added to a 75T cell culture dish containing Sf9 insect cells (1:30 virus titer), and incubated at 27° C. for 5 days. The supernatant containing high concentration of recombinant baculovirus was collected by centrifuging the cell culture at 4° C. at 500×g for 10 min, and stored at 4° C.

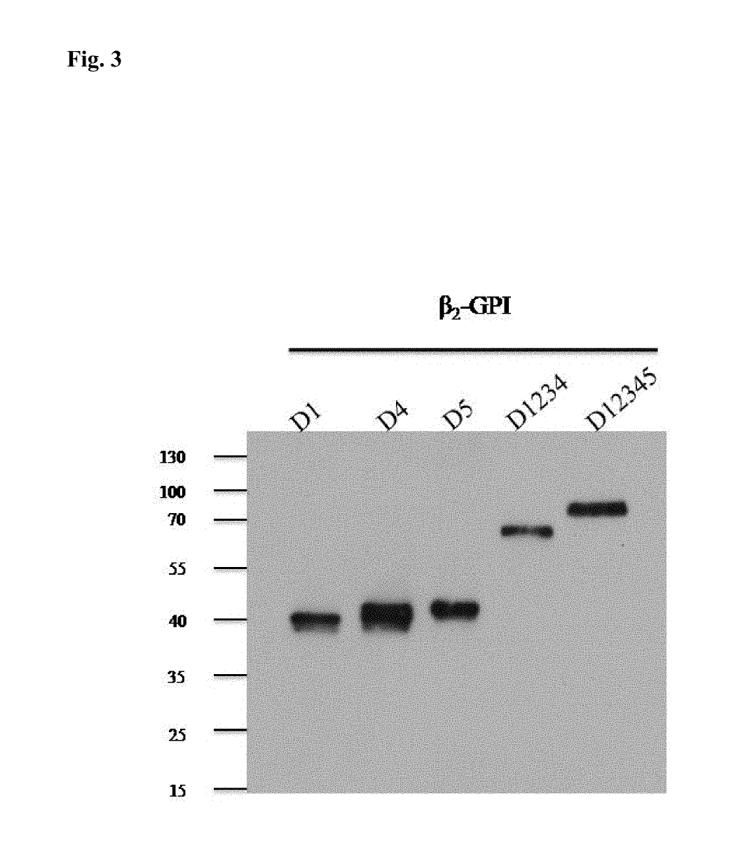

The sf9 insect cells were inoculated in a 6-well cell culture dish, and cultured to about 70% confluent, then infected with the P3 baculovirus for 60 hrs. Finally, the recombinant β2-GPI proteins were purified from the cultured medium of sf9 insect cells infected with baculovirus, and assayed directly by SDS-PAGE and then analyzed by Western blot using anti-strep monoclonal antibody. From the results of Western blot as shown in FIG. 3, recombinant β2-GPI proteins containing various predetermined β2-GPI fragments β2-GPI-D12345, β2-GPI-D1234, β2-GPI-D1, β₂-GPI-D4-Fc and β2-GPI-D5 have been successfully expressed in sf9 insect cell system.

Preparation of the recombinant β2-GPI proteins β₂-GPI-D1-Fc, β₂-GPI-D4-Fc, β₂-GPI-D5-Fc, β₂-GPI-D1234-Fc and β₂-GPI-D12345-Fc

As shown in FIG. 2, in the construct of recombinant β2-GPI bacmids a human IgG1 Fc fusion protein (Fc) was linked to the C-terminus of a predetermined β2-GPI fragment for further purification of recombinant β₂-GPI proteins by protein A affinity chromatography. 200 μl of protein A affinity gel was settled in a 15 ml centrifuge tube, 5 ml of 1×TBS buffer was added and mixed thoroughly by pipetting. The protein A affinity gel was added into a glass column at 4° C. and washed with 5 ml of 50 mM Sodium phosphate/0.75M NaCl (pH7.2) high salt solution. After washing, 5 ml 1×TBS buffer (pH 7.2) was added to balance the column.

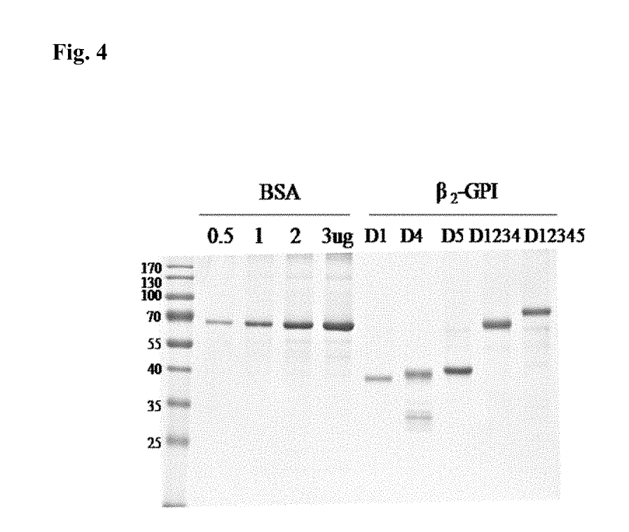

Conditional media of Sf9 cells containing the recombinant peptide domains of β₂-GPI were loaded to the protein A affinity column. The Fc-containing protein was then eluted by acid elution with 0.1M glycine-HCl (pH 3.5) at a flow speed of 1 ml/min. The 5 fractions (each from 0.5 ml elution) were collected, and immediately titrated with 100 μl of Sodium Phosphate buffer (pH 7.2) and vortex to neutralize the acidity of glycine. The purity and yield of the recombinant proteins β₂-GPI-D1-Fc, β₂-GPI-D4-Fc, β₂-GPI-D5-Fc, β₂-GPI-D1234-Fc and β₂-GPI-D12345-Fc were determined by 12% SDS-PAGE, and shown in FIG. 4.

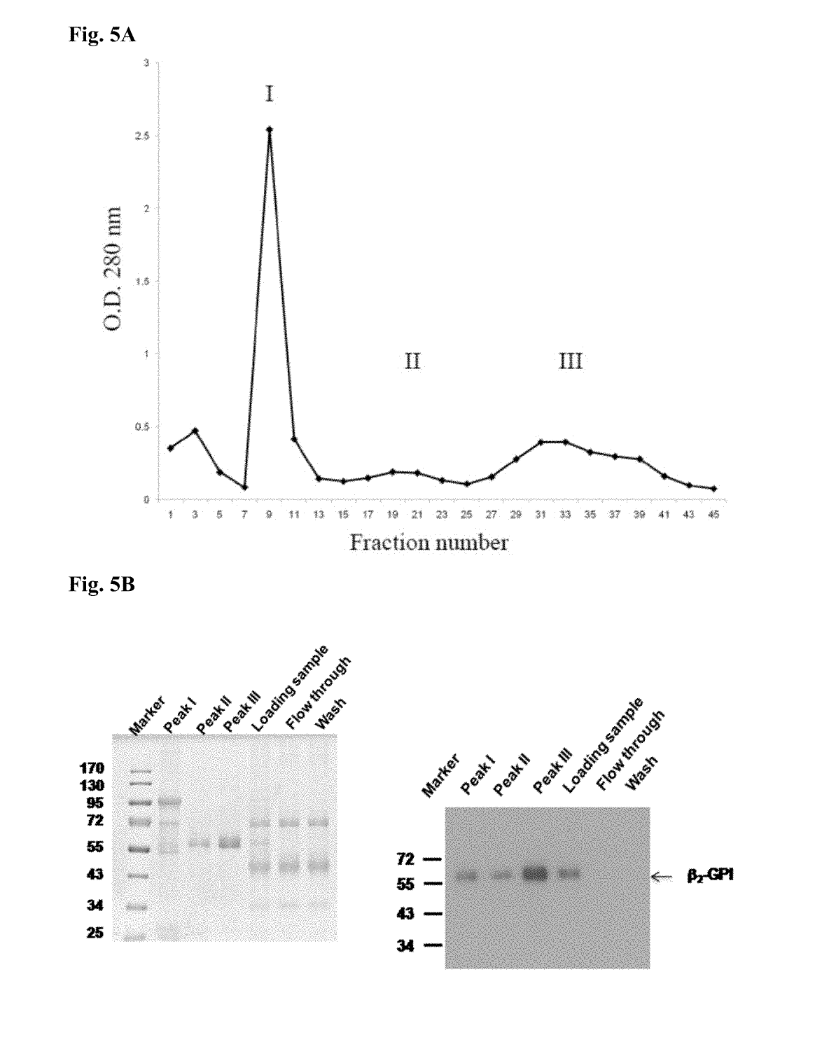

Additionally, the human β₂-GPI purified from plasma was used as a reference and control. The plasma proteins in healthy human plasma were precipitated by perchloric acid, and dialyzed with Tris-base buffer to remove phosphate. The β₂-GPI was isolated by Heparin-Sepharose affinity chromatography using 0.2 M NaCl buffer as the elutant. The absorbance of each collected fractions was determined at 280 nm. FIG. 5A shows the diagram of eluted proteins from Heparin-Sepharose affinity column. The β2-GPI protein was mainly concentrated at peak III as confirmed by SDS-PAGE and western blot analysis (FIG. 5B). The purified β₂-GPI was showed a single band above molecular weight of 55 k Da on the western blot.

### Example 2. Inhibitory Effects of Recombinant β₂-GPI Peptides on B16-F10 Cell Viability and in B16-F10 Melanoma Cells Proliferation and Migration

The effects of various recombinant β₂-GPI peptide fragments β₂-GPI-D1, β₂-GPI-D4, β₂-GPI-D5, β₂-GPI-D1234 and β₂-GPI-D12345 on B16-F10 cell viability and proliferation in B16-F10 melanoma cells were assessed by MTT assay, BrdU proliferation assay and counting the cell number.

B16-F10 melanoma cells were pre-treated with plasma β₂-GPI (200 μg/ml), BSA (negative control, 200 μg/ml), Fc (250 nM) and the recombinant β₂-GPI proteins β₂-GPI-D1, β₂-GPI-D4, β₂-GPI-D5, β₂-GPI-D1234 and β₂-GPI-D12345 (250 nM) for 48 hours, then cell viability was determined by the MTT assay. Briefly, the culture medium was removed after the treatment. The cells were washed with PBS once, then transferred to 100 μl of 0.5 mg/ml MTT (3-[4,5-dimethylthiazol-2-yl]-2,5-diphenyl tetrazolium bromide, Sigma) solution in the dark and incubated at 37° C. for 3 hrs. After incubation, 100 μl of isopropanol was added in the dark and reacted on a shaker for 30 min, then vigorously mixed manually and reacted on a shaker for further 30 min. the absorbance (OD value) of final reactant was determined at 550 nm and 690 nm on an ELIAS reader. The net value was obtained by subtracting the 690 nm absorbance from the 550 nm absorbance. The values were presented as percentage of the control. For counting the cell number, B16-F10 melanoma cells were pre-treated with plasma β₂-GPI (200 μg/ml), BSA (200 μg/ml), Fc (250 nM) and different peptide domains of β₂-GPI (250 nM) for 24, 36 and 48 hours. Cells were trypsinized and counted.

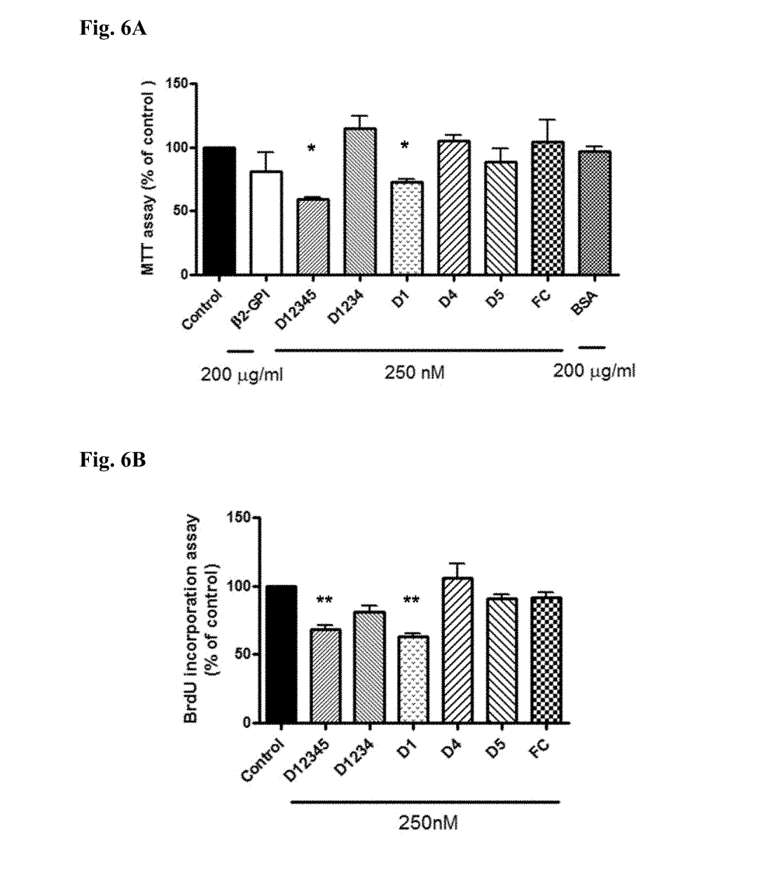

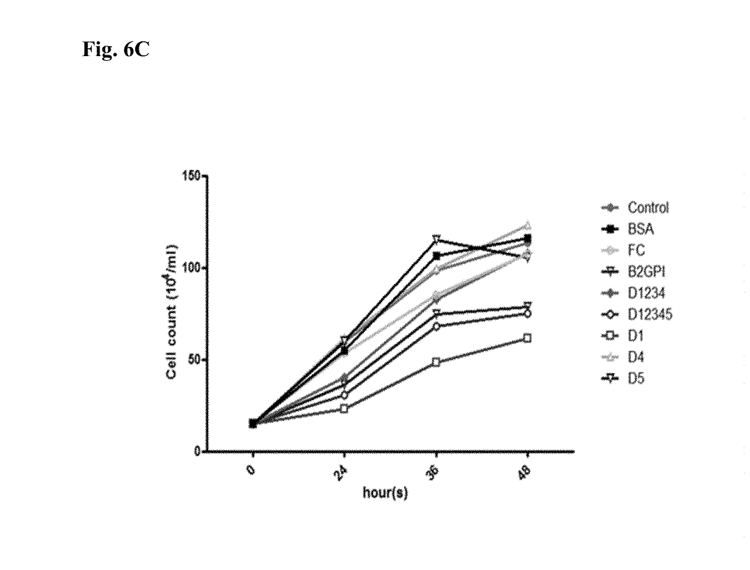

As shown in FIG. 6A, the recombinant β₂-GPI proteins β₂-GPI-D4, β₂-GPI-D5 and β₂-GPI-D1234 have no effects on the cell viability and growth of B16-F10 cells, while the recombinant β₂-GPI-D1 and β₂-GPI-D12345 exhibit comparative or even better inhibitory effects on B16-F10 cells, when compared to the control group. Further, similar results were obtained in the BrdU cell proliferation assay (FIG. 6B) and the cell counting tests (FIG. 6C).

We further evaluated the anti-tumor effect of these β2-GPI recombinant peptides on B16-F10 cell migration by using a Modified Boyden chamber assay. 1.5×10⁵ B16-F10 melanoma cells were inoculated in a 6-well culture dish and cultured overnight. After removing the culture medium and washing with PBSonce, the cells were treated with plasma β₂-GPI (0, 200 μg/ml), BSA (200 μg/ml), Fc (250 nM) and various recombinant β₂-GPI peptides (250 nM), all prepared in an antibiotic free medium containing 2% FBS for 48 hours, then trypsinized and plated (3×10⁴ B16-F10 cells, prepared in an antibiotic free medium containing 2% FBS) into the upper chambers of transwell (Falcon cell culture insert; BD Biosciences) filled with 700 μl medium containing 10% FBS. 24 hours later, cells that had migrated through the filter were collected, fixed by 4% paraformaldehyde and stained with 1% crystal violet for 20 min, then observed and counted under a microscope. The representative photographs are shown with 200× magnification. The number of migrated cells were calculated and presented as percentage of the control.

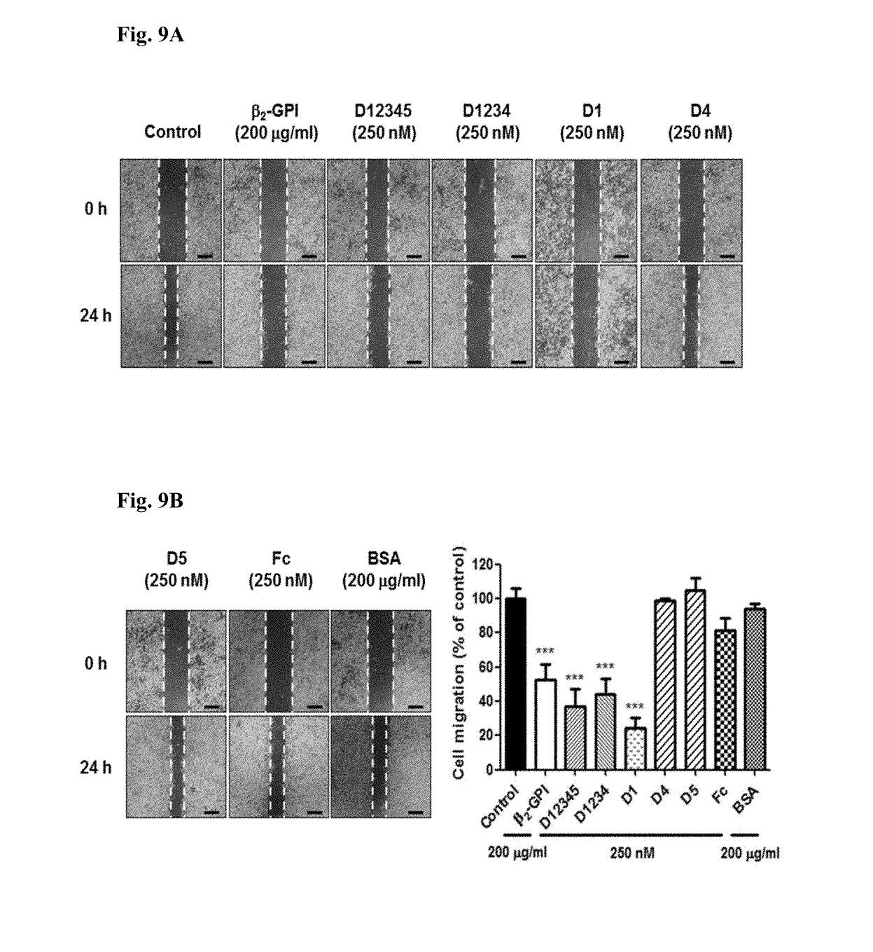

The effects of recombinant β₂-GPI peptides on cell migration in B16-F10 melanoma cells were also evaluated by a wound-healing assay. 1.8×10⁵ of B16-F10 melanoma cells cultured in a 6-well culture dish overnight were pre-treated with or without β₂-GPI (200 g/ml, 4000 nM), β₂-GPI-D12345 (250 nM), β₂-GPI-D1234 (250 nM), β₂-GPI-D1 (250 nM), β₂-GPI-D4 (250 nM) or β₂-GPI (200 μg/ml, 4000 nM) for 48 hours. Similarly, the treatments of Fc fusion tag (250 nM) and BSA (200 μg/ml) were performed as negative controls. Then, cell migration was determined by wound healing assay. After wound induction (white dotted lines indicate the scratched edges), photographs were taken at 0 and 24 hr (FIG. 9A). The wound areas (wound areas of 0 hr−wound areas of 24 hr) were calculated by Image J, and the bar graphs were presented as percentage of control (FIG. 9B).

The effects of plasma purified β₂-GPI (25, 50, 100, 200 μg/m) and the recombinant β₂-GPI peptides (β₂-GPI-D1, β₂-GPI-D4, β₂-GPI-D5, β₂-GPI-D1234 and β₂-GPI-D12345) on B16-F10 cell invasion were further performed by a Matrigel invasion assay. After treating with plasma β₂-GPI, BSA (200 μg/ml), Fc (250 nM) and different peptide domains of β₂-GPI (250 nM) for 48 hours, B16-F10 cells were trypsinized and plated (1.5×10⁵ cells) in the upper chamber of a transwell invasion chamber coated with Matrigel, with a lower chamber containing 10% FBS medium. After incubating for 24 hours, cells that had migrated through the filter were stained and counted. The representative photographs are shown with 200× magnification. The number of invasive cells were calculated and presented as the percentage of the control. Results are means±SEM of at least three independent experiments.

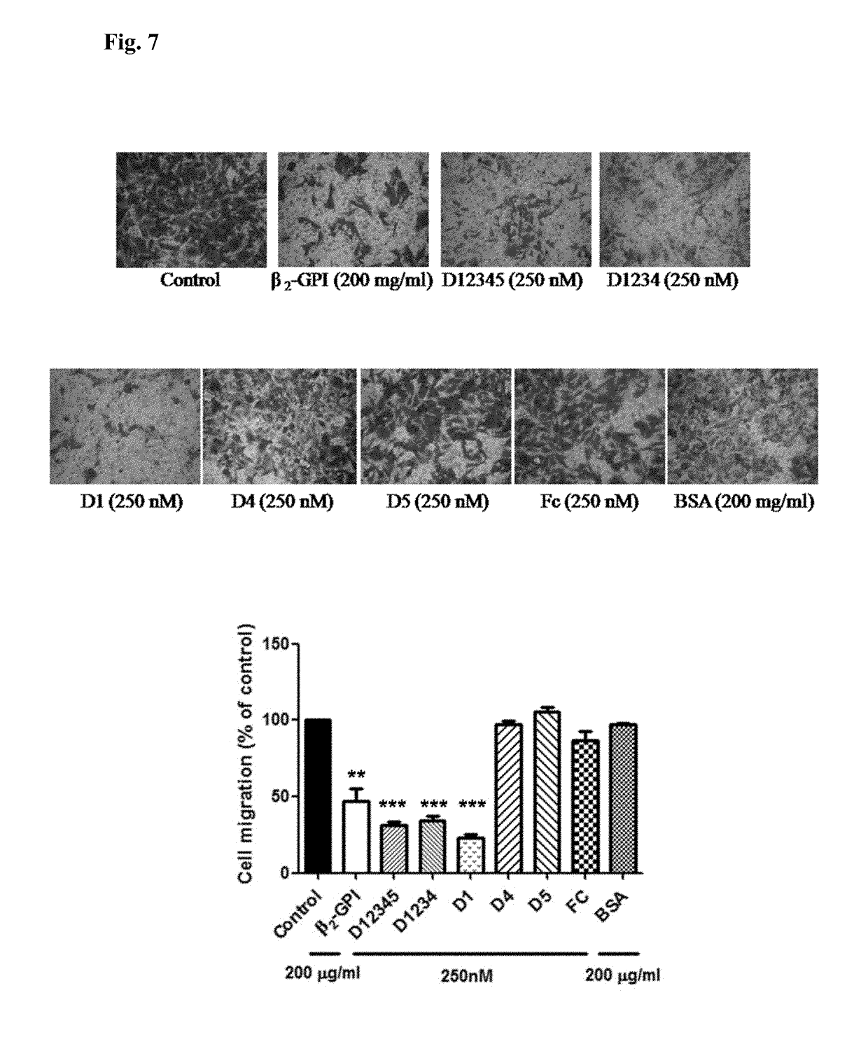

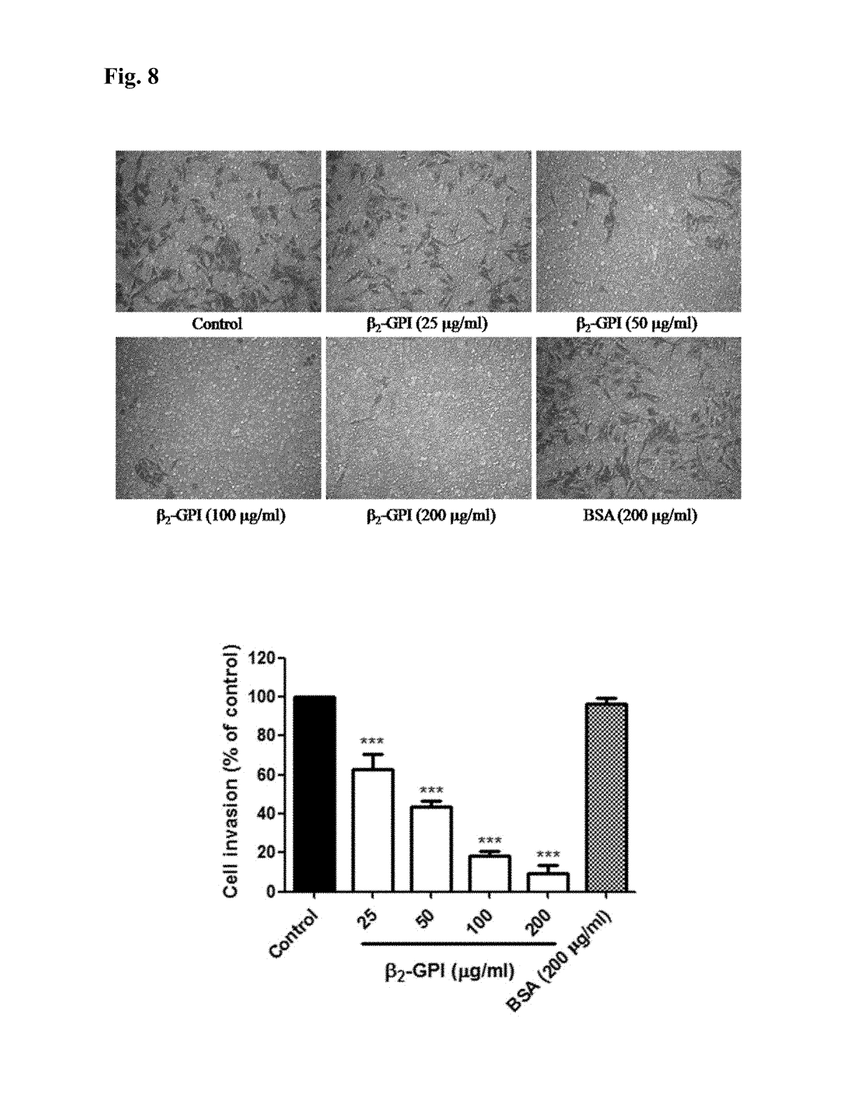

From the results shown in FIG. 7 to 10, it is indicated that recombinant β₂-GPI peptides β₂-GPI-D1, β₂-GPI-D1234 and β₂-GPI-D12345 exhibit similar inhibitory abilities on the cell migration of B16-F10 as compared to purified plasma β₂-GPI, and the β₂-GPI-D1 peptide shows the best inhibitory effects on melanoma cell migration and invasion.

### Example 3. Preparation of Recombinant β₂-GPI Peptides in Lentivirus Expression System

In this example, we designed five constructs of β2-GPI, including D1, D12, D123, D1234 and D12345, and delivered them to A375 and B16-F10 melanoma cells by lentivirus expression system. The widest use of lentivirus is to introduce short-hairpin RNA (shRNA) for decreasing the expression of a specific gene. Additionally, lentiviruses can deliver a significant amount of viral RNA into the DNA of human or animal host cells. For example, the condition of hemophilia in mice can be improved by introducing wild-type platelet factor VIII gene into the diseased mice.

Synthesis of Functional β₂-GPI Peptides

Firstly, the coding sequences of β2-GPI D1, D12, D123, D1234 and D12345 were amplified by PCR using the primers:

5-NheI-GPI-Sense:
(SEQ ID No. 15)
5-GTGCTAGCATGATTTCTCCAGTGCTCATC-3
and
3-EcoRI-D2-GPI-Antisense:
(SEQ ID No. 16)
5-GAATTCCTACTTGTCATCGTCATCCTTGTAGTCTGTACATTTCAGAG
TGTTGATGGG-3,
for pLKO-GPI-FLAG(D1);
(SEQ ID No. 17)
5-GTGCTAGCATGATTTCTCCAGTGCTCATC-3
and
3-EcoRI-D2-GPI-Antisense:
(SEQ ID No. 18)
5-GAATTCCTACTTGTCATCGTCATCCTTGTAGTCAGCACAGACAGGAA
GCTC-3,
for pLKO-GPI-FLAG (D12);
5-NheI-GPI-Sense:
(SEQ ID No. 19)
5-GTGCTAGCATGATTTCTCCAGTGCTCATC-3
and
3-EcoRI-D3-GPI-Antisense:
(SEQ ID No. 20)
5-GAATTCCTACTTGTCATCGTCATCCTTGTAGTCCCTGCATTCTGGT
AATTTAGTCC-3,
for pLKO-GPI-FLAG(D123);
5-NheI-GPI-Sense:
(SEQ ID No. 21)
5-GTGCTAGCATGATTTCTCCAGTGCTCATC-3
and
3-EcoRI-D4-GPI-Antisense:
(SEQ ID No. 22)
5-GAATTCCTACTTGTCATCGTCATCCTTGTAGTCTTTACAACTTGGCAT
GGCAGACC-3,
for pLKO-GPI-FLAG(D1234);
5-NheI-GPI-Sense:
(SEQ ID No. 23)
5-GTGCTAGCATGATTTCTCCAGTGCTCATC-3
and
3-EcoRI-GPI-Antisense:
(SEQ ID No. 24)
5-GAATTCCTACTTGTCATCGTCATCCTTGTAGTCGCATGGCTTTACATC
GGATGC-3,
for pLKO-GPI-FLAG(D12345).

The determined amount of PCR product was mixed with 3 μl Vector pTZ57R/T (preferably, the ratio of PCR product to vector being 3:1), 6 μl 5× Ligation Buffer, 1 μl T4 DNA ligase, provided in the InsTAclone™ PCR Cloning Kit #K1213, and nuclease-free water to final volume of 30 μl. The mixture was incubated at RT for 1 hour or at 16° C. overnight. The ligated DNA was transformed into DH5αcompetent cells.

Preparation of Recombinant Lentivirus for Expressing Various β₂-GPI Peptides

1×10⁶ 293T cells were inoculated in a 6-cm cell culture dish and cultured for 16-18 hours (to about 70-80% confluent) before performing transfection with TurboFect. The culture medium was replaced with 4 ml of fresh RPMI medium. The prepared plasmid DNA and TurboFect transfection reagent were added to a 1.5 ml eppendorf tube containing 400 μl of serum free RPMI medium, mixed thoroughly, and incubated at RT for 15-20 minutes.

Overexpression of Recombinant β₂-GPI Peptides in Melanoma Cells

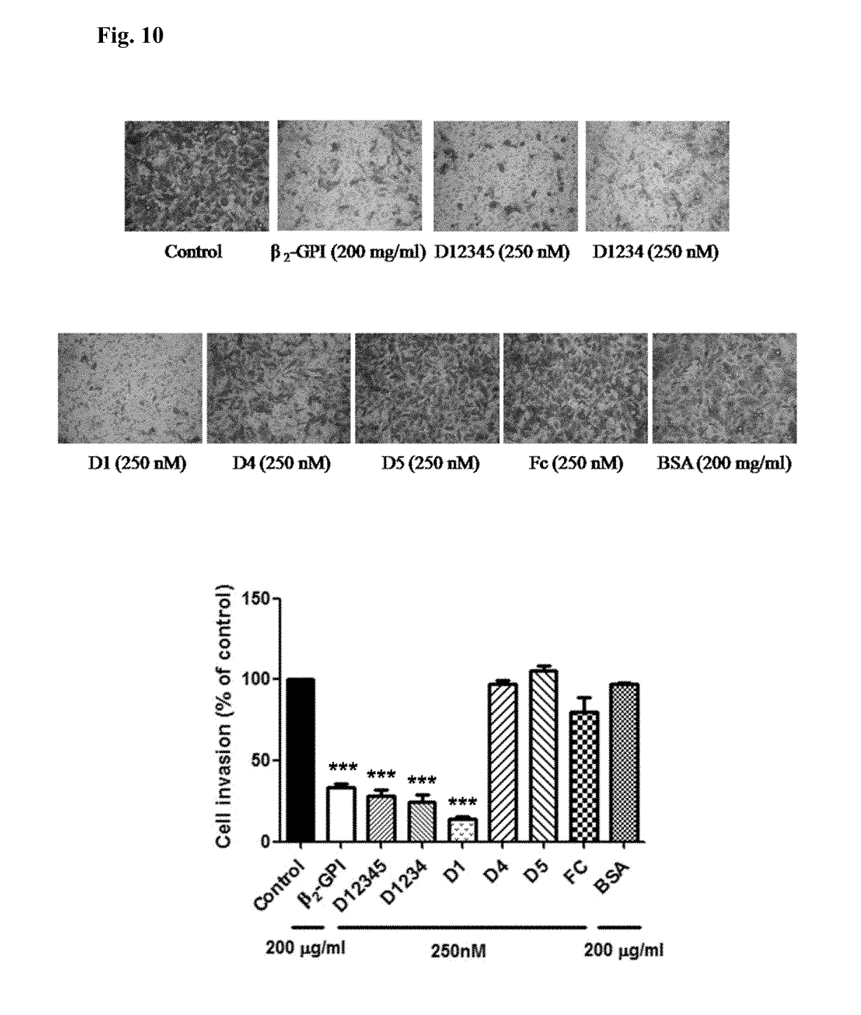

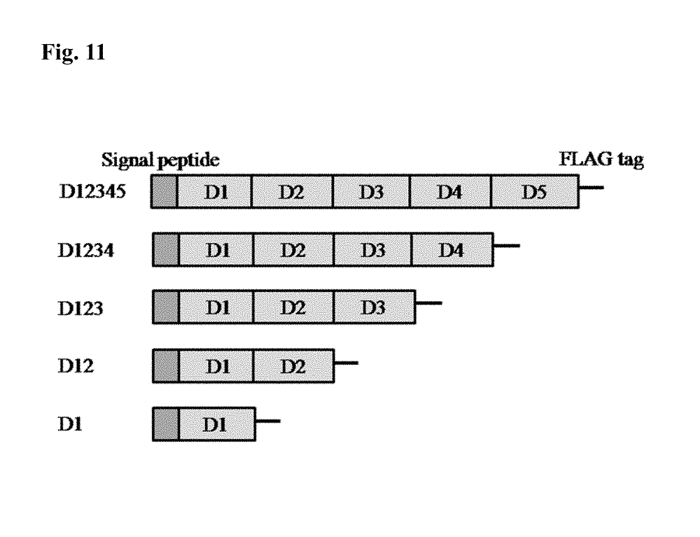

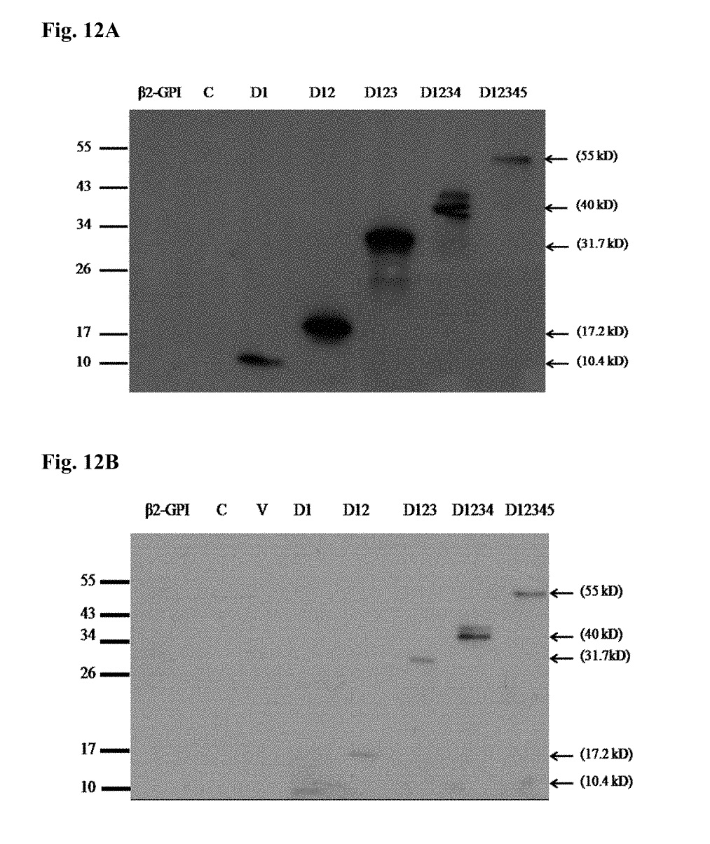

The recombinant lentivirus carrying various DNA fragment coding β₂-GPI peptides β₂-GPI-D1-Flag, β₂-GPI-D12-Flag, β₂-GPI-D123-Flag, β₂-GPI-D1234-Flag or β₂-GPI-D12345-Flag (listed in FIG. 10) was used to infect melanoma cell line A375 and B16-F10 for overexpressing the recombinant β₂-GPI peptides. Briefly, 2.5×10⁵ A375 cells and 2×10⁵ B16-F10 cells were inoculated in a 6-cm cell culture dish, and cultured for 16 hours (to about 30% confluent). The DMEM medium was replaced with 1 ml of medium containing 8 μg/ml of Protamine sulfate (PS), and incubated at RT for 15-20 minutes. 1 ml of viral solution was added into the culture dish, and incubated in a cell culture incubator in P2 laboratory for 24 hours. Then, the culture medium were replaced with fresh DMEM medium, and cultured for further 24 hours.

The infected A375 and B16-F10 melanoma cells by lentivirus packaged with pLKO AS3w.puro β₂-GPI constructs carrying puromycin resistant gene were selected by puromycin (1 μg/ml in A375 cells; 3 μg/ml in B16-F10 cells). After 3 days, the medium was replaced with fresh medium containing halved concentration of puromycin, and the selection was continued for 3 days. Then, the medium was replaced with normal medium, and the cells were cultured for two generations. The cellular protein or RNA was collected and analyzed by western blot or RT PCR to confirm the overexpression of indicated genes.

As shown in FIG. 11, the recombinant β₂-GPI peptides were overexpressed in A375 melanoma cells (FIG. 11A), while host cells without virus infection (control group, C) showed no expression of recombinant β₂-GPI peptides. The B16-F10 melanoma cells infected by lentivirus packaged with pLKO AS3w.puro β₂-GPI constructs also showed the overexpression of recombinant β₂-GPI peptides (FIG. 11B).

### Example 4. Tumor Growth Inhibiting Effects of Recombinant β₂-GPI Peptides in B16-F10 Implanted C57BL/6 Mice

From the previously described results, it is indicated that the recombinant β₂-GPI-D1, PI-D1234 and β₂-GPI-D12345 exhibit better inhibitory effects on B16-F10 cell proliferation and migration, when compared to the other recombinant β₂-GPI peptides. In this example, a model of subcutaneous injection of tumor cells in C57/BL6 mice was used to identify the functional domain of β₂-GPI in inhibiting tumor growth in vivo.

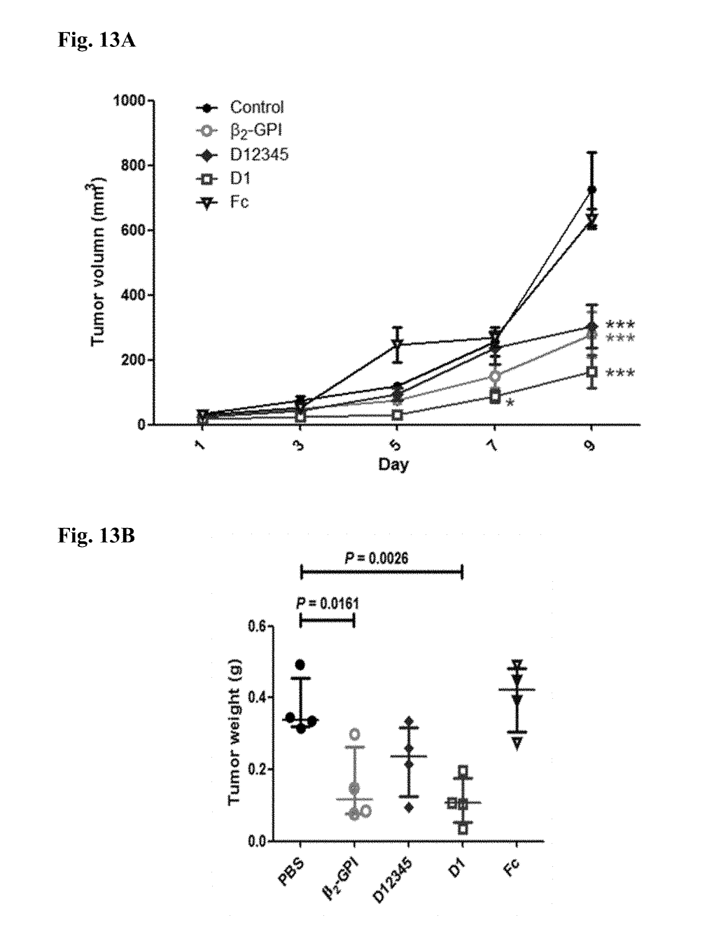

5×10⁵ B16-F10 melanoma cells were injected into the dorsal flank of 6 to 8 week-old C57BL/6 mice. Around 5 days after injection, the solid tumor size had reached to about 30 mm³. The mice were randomized into 5 groups (n=4), and then treated with purified β₂-GPI (300 μg, 24 μM/day, 12 mg/kg/day, 1.2 mg/ml/day), recombinant β₂-GPI peptides β₂-GPI D12345 (300 μg, 15 μM/day, 12 mg/kg/day, 1.2 mg/ml/day) and β₂-GPI D1 (146.25 μg, 15 μM/day, 5.85 mg/kg/day, 0.585 mg/ml/day), or Fc (131.25 μg, 15 μM/day, 5.25 mg/kg/day, 0.525 mg/ml/day) by subcutaneous injection for 9 days. Tumor volume was measured with a vernier caliper every two days and calculated using the formula: Tumor volume (mm³)=length (mm)×width² (mm²)×0.5. Results are shown in FIG. 13A.

The results indicate that tumor growth was significantly inhibited in the mice treated with purified β₂-GPI, recombinant β₂-GPI peptides f₂-GPI D12345 and β₂-GPI D1 (FIG. 13A) for 9 days, with the tumor volume reduced to 38.4%, 41.8% and 22.5% of control group, respectively.

At the day 10 of treatment, mice were sacrificed and tumors were isolated and weighted. As shown in FIG. 13B, tumor weight in the mice treated with purified β₂-GPI protein, recombinant β₂-GPI D12345 and β₂-GPI-D1 peptides were significantly reduced to 0.15 g, 0.23 g and 0.11 g, respectively, when compared to the tumor in the control group treated with PBS (0.37 g). Therefore, the tumor growth (in both size and weight) was inhibited by purified β₂-GPI protein, recombinant β₂-GPI D12345 and β₂-GPI-D1 peptides.

Base on the results described in the examples, recombinant β₂-GPI peptides of the invention exhibit suppressive activities on the proliferation and migration of tumor cells, which promise the application of the recombinant β₂-GPI peptides in producing medicines for treating or preventing tumor formation, tumor cell proliferation and metastasis. In comparison to purified plasma β₂-GPI protein, recombinant β₂-GPI peptides of the invention are more potential for the development of anti-tumor protein drugs for they only contain at least one functional fragment of β₂-GPI, and may be produced in eukaryotic cells by using viral expression system.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A purified recombinant β₂-GPI peptide, comprising at least one functional β₂-GPI peptide fragment exhibiting anti-tumor activities of inhibiting tumor cell proliferation, migration and/or angiogenesis.

2. The purified recombinant β₂-GPI peptide of claim 1, wherein the functional β₂-GPI peptide fragment is selected from a group consisted of a domain 1 (D1, SEQ ID No. 2), domain 2 (D2, SEQ ID No. 3), domain 3 (D3, SEQ ID No. 4), domain 4 (D4, SEQ ID No. 5), domain 5 (D5, SEQ ID No. 6) of β₂-GPI protein.

3. The purified recombinant β₂-GPI peptide of claim 1, wherein the purified recombinant β₂-GPI peptide comprises a domain 1 (D1) functional peptide fragment of β₂-GPI protein.

4. The purified recombinant β₂-GPI peptide of claim 1, wherein the purified recombinant β₂-GPI peptide comprises a domain 4 (D4) functional peptide fragment of β₂-GPI protein.

5. The purified recombinant β₂-GPI peptide of claim 1, wherein the purified recombinant β₂-GPI peptide comprises domain 1 (D1) and domain 4 (D4) functional peptide fragments of β₂-GPI protein.

6. A pharmaceutical composition for suppressing tumors, comprising the purified recombinant β₂-GPI peptide claim 1, and a pharmaceutical acceptable carrier, excipient or diluent.

7. The pharmaceutical composition of claim 6, wherein the purified recombinant β₂-GPI peptide comprises a domain 1 fragment (D1, SEQ ID No. 2) of β₂-GPI protein.

8. The pharmaceutical composition of claim 6, wherein the purified recombinant β₂-GPI peptide comprises a domain 4 fragment (D4, SEQ ID No. 5) of β₂-GPI protein.

9. The pharmaceutical composition of claim 6, wherein the purified recombinant β₂-GPI peptide comprises a domain 1-4 fragment (D1234, SEQ ID No. 7) of β₂-GPI protein.

10. The pharmaceutical composition of claim 6, wherein the purified recombinant β₂-GPI peptide comprises a domain 1-5 fragment (D12345, SEQ ID No. 8) of β2-GPI protein.

11. The pharmaceutical composition of claim 6, wherein the pharmaceutical composition is used to inhibit tumor cell growth.

12. The pharmaceutical composition of claim 6, wherein the pharmaceutical composition is used to inhibit angiogenesis in a tumor progressing site.

13. The pharmaceutical composition of claim 6, wherein the pharmaceutical composition is used to inhibit tumor metastasis.
